(19) United States
(12) Patent Application Publication
(10) Pub. No.: US 2026/0248574 A1
(43) Pub. Date: Aug. 27, 2026

(54) SURGERY SYSTEM

(71) Applicant: Erbe Elektromedizin GmbH, Tuebingen (DE)

(72) Inventors: Peter SELIG, Nehren (DE); Walter LINZENBOLD, Boeblingen (DE); Michael EDERER, Stuttgart (DE)

(21) Appl. No.: 19/535,341

(22) Filed: Feb. 10, 2026

(30) Foreign Application Priority Data
Feb. 25, 2025 (EP) 25159859.5

(51) Int. Cl. A61B 34/37; A61B 18/00; A61B 18/12; A61B 18/14; A61B 34/00; A61B 34/20

(52) U.S. Cl. CPC A61B 34/37; A61B 18/1233; A61B 18/14; A61B 34/20; A61B 34/74; A61B 2018/00601

(57) ABSTRACT

A surgery system comprising a device and a robot as well as a method for the operation thereof are disclosed. The device comprises a supply apparatus and at least one instrument for treating biological tissue connected to the supply apparatus. The instrument is arranged on a holding device of the robot and can be positioned and/or moved relative to the tissue to be treated using the robot, wherein this handling is described by at least one handling parameter. At least one of the handling parameters or multiple handling parameters are provided to the device that depending on one or more handling parameters determines and controls in open-loop or closed-loop manner one or more operation parameters for the operation of the instrument. In doing so, an adaptation of the operation of the instrument to the current movement of force exerted on the instrument by means of the robot is achieved.

### CROSS REFERENCE TO RELATED APPLICATION

This application claims the benefit of European Patent Application No. 25159859.5, filed Feb. 25, 2025, which is incorporated herein by reference in its entirety.

### TECHNICAL FIELD

The invention refers to a surgery system having a robot and a device. The device has a supply apparatus as well as an instrument connected to the supply apparatus, wherein the instrument can be an electrosurgical instrument, for example, or any other instrument that is supplied by the supply apparatus with at least one operating medium (for example gas and/or liquid and/or ultrasound and/or light and/or electrical power). The instrument serves for treatment of biological tissue of a patient.

The robot has a robot arm with a holding device arranged on the robot arm. The holding device is configured for releasably arranging an instrument or as an option also multiple instruments. By means of the robot the at least one instrument can be positioned and/or moved relative to the biological tissue and/or a force can be created between the instrument and the biological tissue.

### BACKGROUND

Such surgery systems having a robot that handles or guides the instrument during treatment can simplify the treatment of biological tissue for an operator (for example surgeon). For example, WO 2014/004116 A1 discloses a surgery system having a robot and an electrosurgical instrument that is held and moved by the robot. A robot control can thereby adapt the speed of the instrument to a tissue type that is to be treated, wherein the tissue type can be particularly determined based on a determined impedance.

It is known from EP 2 854 665 A2 to measure measurement values, for example a force or a speed of an instrument relative to a treated tissue, and to determine and set the desired tissue treatment type based on such handling parameters. For this purpose, respective sensors are provided on the electrosurgical instrument.

In EP 3 795 106 B1 a token-based control is described. Using a token a robot control of a robot and a control of an HF generator supplying an electrosurgical instrument are coordinated with each other.

EP 4 132 407 A2 relates to a surgery system in which the clamping force of a forceps-like instrument guided by a robot arm can be set depending on the impedance of the clamped tissue.

### SUMMARY

Different approaches for coordination of the control of a robot and the operation of an instrument held on the robot are thus described in the prior art. For such coordination it is important that the handling of the instrument by means of the robot and the operation of the instrument for treatment of biological tissue are very precisely coordinated with each other, in order to achieve the desired treatment effect. For example, it has been proposed for this purpose to arrange additional sensors on the instrument in order to obtain information about the actual position or movement of the instrument or the force applied by the instrument on the tissue. Additional sensors do not only increase the costs and the number of components of such a surgery system, but require in addition installation space that can in turn limit the possible applications of the instrument. Additional sensors can make handling of the instrument more difficult or can hinder the handling of the instrument during the treatment of biological tissue.

It can be considered as one object of the present invention to provide a surgery system that guarantees a very good temporal coordination between the positioning and/or movement of the instrument on one hand and the supply of the instrument on the other hand and particularly guarantees a coordination in real time or with little latency, while providing a simple construction.

This object is solved by means of a surgery system and a method as described herein.

The surgery system according to the invention comprises a device. The device has a supply apparatus having an apparatus control. At least one electrosurgical instrument is connected or can be connected to the supply apparatus. The supply apparatus can supply the connected instrument or at least one of the connected instruments with at least one operation medium required for the operation, for example with electrical power or energy. Additionally or alternatively, the supply apparatus can also supply a fluid (liquid, gas) or ultrasound power or light as an operation media required for the operation of one or more connected instruments (for example gas for plasma creation or water for a cryoinstrument, etc.) These operation media can be provided in arbitrary combination.

The apparatus control of the supply apparatus is configured to determine and set (open-loop or closed-loop control) at least one operation parameter for the connected instrument or for at least one of the connected instruments. Depending on the used instrument, for example, the at least one operation parameter can be an electrical operation parameter and/or fluidical operation parameter and/or ultrasound operation parameter and/or light operation parameter, etc. Depending on the instrument configuration, the connection between the supply apparatus and the at least one connected instrument can be an electrical and/or fluidical and/or optical and/or ultrasound guiding connection. For example, by means of an optical connection, light can be transmitted (for emission on the instrument and/or light detected by a sensor on the instrument).

Each provided electrosurgical instrument can have, for example, one electrode, two electrodes or also more than two electrodes and can accordingly be configured as monopolar, bipolar or multipolar instrument. To the at least one electrode in accordance with an electrical operation parameter, an electrical voltage can be applied and/or a current can flow into or via the electrode, if a treatment circuit is closed.

If the electrosurgical instrument is a monopolar instrument or is used as a monopolar instrument, a neutral electrode that is to be attached to a patient can be connected to the supply apparatus. In this case the treatment circuit can be closed from the supply apparatus via the electrosurgical instrument or its electrode, the biological tissue to be treated of the patient, the neutral electrode and from there back to the supply apparatus. If the electrosurgical instrument is a bipolar or a multipolar instrument, the treatment circuit can be closed from the supply apparatus via one electrode of the electrosurgical instrument, the biological tissue to be treated, another electrode of the electrosurgical instrument and back to the supply apparatus.

The surgery system comprises in addition a robot that is configured for handling the instrument or at least one of the provided instruments. Here, handling means:
a) the positioning and/or moving of the instrument relative to biological tissue or relative to the patient; and/or
b) the application of a force between the instrument and the biological tissue to be treated, for example if by means of the instrument a clamping force and/or a pressing force and/or a cutting force shall be applied on the biological tissue to be treated.

The robot comprises a holding device for holding the instrument or the at least one instrument. The holding device is arranged on a movable robot arm of the robot.

The handling is characterized by at least one handling parameter. The at least one handling parameter is preset in the robot or is determined in the robot, for example in the robot control or by means of the robot control.

For determination of the at least one handling parameter outside the robot no additional sensors are provided on the instrument that is held by the robot. Such additional sensors mean particularly sensors that are not anyway present and used in the robot for the movement control of the robot arm, but are exclusively assigned to an instrument arranged on the robot.

The at least one handling parameter does not comprise operation parameters for the operation of the at least one instrument arranged on the holding device. The handling parameter or at least one of the handling parameters or all of the handling parameters can be determined using electrical control parameters and/or electrical robot sensor values only, which are used anyway and are thus available for control of the robot and particularly a drive unit of the robot arm. Such electrical control parameters and/or robot sensor values can be used or can be available in the robot for open-loop or closed-loop control of a drive unit, particularly electric motor drive unit, of the movable robot arm. Thereby each handling parameter can be, for example, a preset set point value for the drive unit or a determined or measured actual value of the drive unit, describing the position, the movement or the applied force on the drive unit of the robot arm.

The surgery system comprises in addition a communication device. The communication device is configured to provide the at least one handling parameter of the robot for the apparatus control. Based on the at least one handling parameter the apparatus control determines the at least one operation parameter for the respective instrument (for example the currently used instrument) to which the at least one handling parameter belongs, for example during a treatment of biological tissue. Based on the at least one operation parameter the respective instrument is then supplied by the supply apparatus. Depending on the type of the operation parameter, the supply apparatus can comprise for this purpose, for example, a generator for producing a generator voltage and/or a generator current and/or it can comprise a fluid source and/or a light source and/or an ultrasound source. The generator and/or the provided sources serving for supply can be open-loop or closed-loop controlled by means of the apparatus control, in order to adjust the at least one operation parameter for the instrument. For example, one of the parameters or multiple of the parameters indicated in the following in arbitrary combination can be used as operation parameter:
an electrical power for the instrument, particularly for the electrode of the instrument;
an activation duration during which an electrical power, and/or an electrical voltage and/or an electrical current is continuously or in clocked manner provided for the instrument, particularly for an electrode of the instrument;
a current parameter of the electrical current for the instrument, particularly for an electrode of the instrument, wherein the current parameter can be an amplitude, a frequency, a crest factor, a duty factor or duty cycle, a waveform or an arbitrary combination of these current parameters;
a voltage parameter for an electrical voltage for the instrument, particularly for an electrode of the instrument, wherein the voltage parameter can be an amplitude, a frequency, a crest factor, a duty factor or duty cycle, a waveform or an arbitrary combination of these voltage parameters;
a fluid parameter of a fluid supplied to the instrument, for example a pressure, a flow velocity, a volume flow rate, a mass flow rate or an arbitrary combination of these fluid parameters;
an ultrasound parameter of an ultrasonic wave provided to the instrument, for example its frequency and/or amplitude, and/or power;
a light parameter of the light provided to the instrument, for example its frequency and/or amplitude and/or power.

The configuration according to the invention allows the quick and adaptive setting of the at least one operation parameter for the operation of the at least one instrument based on present information about the handling of the instrument by means of the robot (characterized by the at least one handling parameter). In doing so, it is known to the apparatus control how the robot positions and/or moves the respective instrument at present and/or whether a force between the instrument and the tissue to be treated occurs and/or the amount of such a force (for example clamping force, pressing force, cutting force).

The apparatus control is able to adapt the at least one (for example electrical and/or fluidical) operation parameter if it should be necessary due to a change of a handling parameter, for example if a force or a movement of the instrument relative to the tissue to be treated changes. Such adaptions can be determined and set in the supply apparatus or the apparatus control very quickly and almost in real time and at least without relevant latency. In doing so, it is guaranteed that the at least one operation parameter is adapted at any point in time with high accuracy to the handling of the instrument described by the at least one handling parameter.

As explained, for determining the at least one handling parameter no additional sensors are required on the instrument. Rather setpoint values, actual values or other parameters are used that are available anyway in the robot or the robot control in order to position and/or move the robot arm or in order to apply a desired force between the instrument held on the robot arm and a tissue to be treated using the robot arm. In doing so, additional construction efforts and additional costs can be avoided. In addition, the required installation space in the area of the instrument is not enlarged by additional sensors, which is advantageous for the positioning and/or movement of the instrument and therefore in turn for the treatment of biological tissue.

In an embodiment the surgery system can comprise an operation device that serves as human-machine interface or as operator interface. Such an operation device can be configured for input and/or output of information and data by an operator and/or to an operator. For this purpose, input devices and/or output devices can be provided, for example a touchscreen, keys, a speaker, a touch-sensible field for operating a pointing device on a screen, a joystick, or any other input devices and/or output devices known per se, for example from the field of machine or robot control.

The communication device can have at least one interface or can be formed by at least one interface. For example, the at least one communication interface or communication device can be part of the apparatus control and/or part of the robot control and/or part of the operation device.

The communication device can transmit data and information in wireless and/or wired manner and for this purpose can comprise the required wireless and/or wired communication interfaces. The communication device is particularly configured to transmit data and information unidirectionally or bidirectionally between the following participants: (i) between the robot control and the apparatus control and as an option (ii) between the operation device and the apparatus control and as an option (iii) between the operation device and the robot control.

The communication device or the at least one communication interface can be configured to operate according to a standard communication protocol. For example, the at least one communication interface can be a USB interface, an Ethernet interface, a CAN interface, a Bluetooth interface, a WLAN interface, a 5G interface, a WiFi interface or an arbitrary combination thereof. In an embodiment the communication can be carried out according to the communication protocol IEEE 11073 SDC (“Service-oriented Device Connectivity”).

It is advantageous if the robot comprises at least one drive unit for positioning and/or moving of the holding device arranged on the robot arm. The at least one drive unit can be particularly configured to position and/or move two arm parts of the robot arm, which are movably connected with one another, relative to one another. For example, such arm parts can be rotated around at least one axis relative to each other or can be pivoted relative to each other. Each drive unit can comprise an electric motor for this purpose, the operation of which carries out the relative movement between the two arm parts. Each drive unit can be individually controlled by the robot control, so that a relative movement between two directly connected arm parts of the robot arm can be carried out independent of a relative movement of two other arm parts that are directly connected with each other.

The operation of the electric motor can be described by at least one motor parameter. A motor parameter can be a preset setpoint value, a measured actual value or another determined actual value. The motor parameter can be, for example, an electric parameter, such as a motor voltage, a motor current, an electrical power of the motor, a frequency of the motor voltage and/or the motor current or the like. Additionally or alternatively, the motor parameter can be a torque of the electric motor and/or a rotational speed of the electric motor and/or a rotation angle of the electric motor and/or a temporal change of any motor parameter indicated above (temporal derivative of any order, for example first order or second order).

The motor parameter, one of the motor parameters or all motor parameters can be determined without using sensors, for example. As an option at least one of the provided drive units can comprise at least one motor sensor, for example in order to determine a motor rotational speed and/or a motor torque by means of sensors. Such a motor sensor is a robot sensor of the robot and not an additional sensor. Apart from such motor sensors, the robot can omit additional sensors. The at least one optionally provided robot sensor is preferably a sensor configured for detection of a relative movement and/or a relative position.

A handling parameter can be directly a setpoint value and/or actual value, for example one of the above-mentioned motor parameters, which is available in the robot. Additionally or alternatively, based on at least one setpoint value and/or actual value or motor parameter at least one handling parameter can be determined using a function, a characteristic curve, a characteristic set of curves, a table (“look-up table”).

In a preferred embodiment the operation parameter for the instrument or for one of the instruments connected to the supply apparatus that is open-loop or closed-loop controlled by the apparatus control can be determined and set depending on a movement speed of the respective instrument relative to the tissue to be treated. For this purpose, for example, an electrical power and/or an electrical current and/or an electrical voltage can be adapted to the movement speed.

In an embodiment, depending on the movement speed of the instrument, the electrical power on the instrument or on at least one electrode of the instrument is changed and thereby particularly increased if the movement speed increases. The power can be changed in one or multiple steps or continuously. It is thereby particularly advantageous if the electrical power on the instrument is changed and the electrical voltage at the at least one electrode of the instrument is thereby maintained constant.

In any embodiment optionally the movement or position of the robot arm can be controlled in open-loop or closed-loop manner based on the at least one determined operation parameter and/or treatment parameter and/or tissue parameter of the tissue to be treated. For example, the robot arm can be stopped in a position or can carry out a defined movement if the instrument has reached a position in which a specific tissue type has been determined, for example tumor tissue. The determination can be carried out based on at least one determined parameter, for example a tissue impedance and/or based on a spectrum of the tissue (optical emission spectroscopy OES). The movement of the robot arm can additionally or alternatively also depend on whether a predefined treatment effect has been achieved.

It can be particularly advantageous to turn off an electrical power provided to the instrument and particularly to the at least one electrode of the instrument if the instrument stands still relative to the treated tissue or if, since the start of the standstill of the instrument relative to the treated tissue, a predefined minimum standstill duration has lapsed.

Additionally or alternatively, the at least one operation parameter can also be changed depending on a force between the respective instrument and the tissue to be treated (for example cutting force and/or pressing force and/or clamping force). The change can be carried out in steps or continuously. For example, depending on a handling parameter describing the force, an operation parameter or multiple operation parameters can be changed. In an embodiment the at least one operation parameter can be changed with increasing force as follows:
the electrical power is increased continuously or in steps with increasing force;
the electrical voltage is increased in steps or continuously with increasing force;
the crest factor is reduced in steps or continuously with increasing force.
   All other electrical operation parameters can thereby be adapted independent from the force and can be maintained constant, for example.

The at least one operation parameter, particularly at least one electrical operation parameter, can be changed depending on a penetration depth or a cutting depth of a tool of a (for example electrosurgical) instrument in the tissue to be treated, particularly as follows:
the electrical power at the electrosurgical instrument can be increased in steps or continuously with increasing penetration depth or cutting depth;
the crest factor can be reduced in steps or continuously with increasing penetration depth or cutting depth.
   All other electrical operation parameters can thereby be set independent from the penetration depth or cutting depth and can be maintained constant, for example.

If the instrument comprises two instrument parts that can be moved relative to each other and particularly pivoted relative to each other, the relative position (for example a relative angle) and/or a relative movement and/or a force between the two movable instrument parts can be determined as at least one handling parameter and depending thereon at least one operation parameter can be changed. Such instrument parts can be, for example, jaws of a forceps-like tool or instrument that can be pivoted relative to each other and/or can be a tool (for example needle or knife) that can be moved inside an instrument channel.

For example, tissue can be clamped between the instrument parts or jaws in order to coagulate and optionally additionally cut through the tissue. Preferably, thereby the operation parameter can be changed depending on a determined clamping force or closing force between two movable instrument parts as follows:
an electrical power on the at least one movable tool part can be reduced in steps or continuously with increasing force between the two tool parts;
an activation duration during which an electrical power is provided to at least one of the movable tool parts is reduced continuously or in steps with increasing force between the two tool parts.
   All other electrical operation parameters can thereby be set independent from the clamping force or closing force between two movable instrument parts and can be maintained constant, for example.

Depending on the relative position or relative angle between the two movable tool parts, at least one of the operation parameters can be changed as follows:
an electrical power on at least one of the movable tool parts can be increased in steps or continuously with increasing relative distance or increasing angle between the tool parts;
an activation duration during which an electrical power is applied to at least one of the movable instrument parts can be increased in steps or continuously with increasing relative distance or increasing angle between the movable tool parts.
   All other electrical operation parameters can thereby be set independent from the relative position or the relative angle between the two movable tool parts and can be maintained constant, for example.

For determination of a contact or reference position relative to the tissue to be treated the point in time of a contact or a touching contact between the instrument part and the tissue to be treated can be determined, for example by means of the apparatus control and/or by means of the robot control. If on an instrument part a voltage is applied, a voltage change upon contact of the instrument part with the tissue to be treated can be determined by means of the apparatus control. Additionally or alternatively, in case of such a contact, a current change of a current flowing into the instrument or the instrument part can be determined. If the tissue to be treated provides sufficient resistance to the penetration of an instrument part, additionally or alternatively, also an increasing motor current in the drive unit of the robot can be determined, which is necessary in order to overcome the increasing counterforce.

This contact or reference position of the instrument held on the holding device with the tissue can be used for determination of a penetration depth or cutting depth into the tissue to be treated, whereby no additional sensor is required. Position change relative to this contact or reference position into the tissue to be treated corresponds to the penetration depth or cutting depth.

The robot and, for example, the holding device can be configured to move two instrument parts, which are movable and, for example, pivotable relative to each other of an instrument held on the holding device. The relative position and/or relative movement and/or force between the movable tool parts can be determined by the robot and particularly by means of the robot control. For example, the instrument parts that are movable relative to each other can be moved in a reference position (for example, completely closed position or completely closed direct contact position). Position changes carried out subsequently to this reference position can be determined in order to determine the present relative position and the relative movement (for example relative speed) without requiring an additional sensor.

A force that is necessary for actuating movable instrument parts by means of the robot or the holding device can be measured and/or determined otherwise in the robot. For example, a motor current in a drive unit that is necessary for the actuation can be characteristic for the closing force or clamping force between the movable instrument parts.

### BRIEF DESCRIPTION OF THE DRAWINGS

Advantageous embodiments of the invention are derived from the dependent claims, the description and the drawing. In the following, preferred embodiments are explained in detail based on the attached drawing. The drawing shows:

FIG. 1 a schematic, block diagram-like illustration of a surgery system comprising a robot and a device,

FIG. 2 a block diagram of an embodiment of a device for the surgery system according to FIG. 1,

FIG. 3 a block diagram of an embodiment having multiple drive units of the robot of FIG. 1, which can be controlled by means of a robot control, and

FIG. 4 a schematic principle illustration in the type of a block diagram to illustrate a relation between the control of the robot and the control of the device carried out depending on the control of the robot.

### DETAILED DESCRIPTION

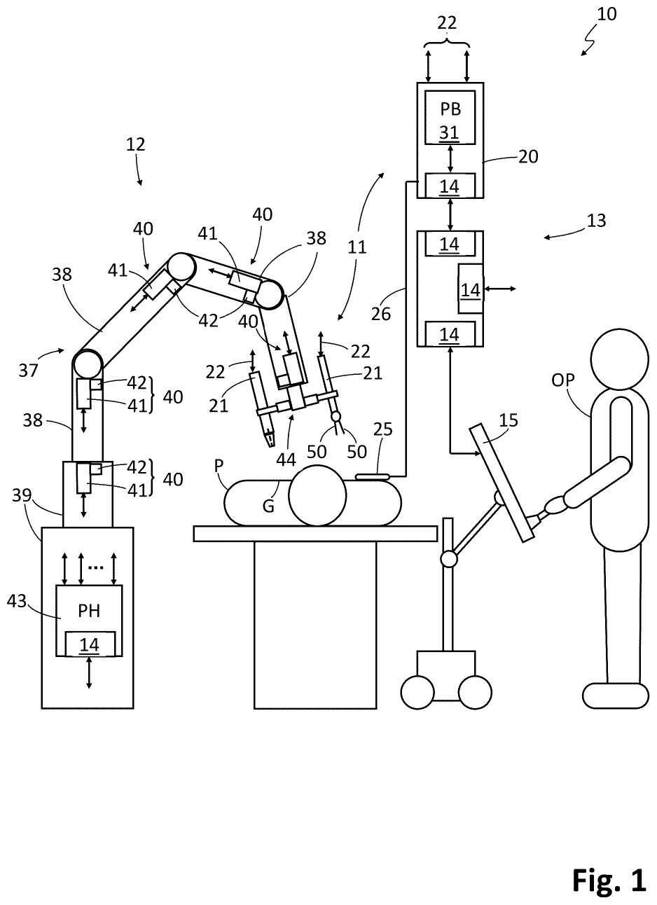

In FIG. 1 an embodiment of a surgery system 10 is illustrated highly schematically in the type of a block diagram. The surgery system 10 comprises a device 11 and a robot 12. The device 11 and the robot 12 are communicatively connected by means of a communication device 13, in order to be able to transmit electrical and/or optical signals, preferably bidirectionally.

In the embodiment the surgery system 10 has additionally an operation device 15 by means of which the surgery system 10 can be operated by an operator OP. The operation device 15 thus represents an operation interface or human-machine interface. The operation device can be configured to input commands and/or output information via suitable input units and/or output units. The operation device 15 can comprise, for example, a screen, a touch-sensible field or screen, movement-controlled elements or the like.

Using the operation device 15 the robot 12 and preferably also the device 11 can be operated and controlled by the operator OP. For example, an operator can select or adjust an application (mode) for the device 11 using the operation device 15 and/or directly on the device 11. An application can be, for example, the type of treatment (for example cutting, coagulating, ablating, etc.) of biological tissue G of a patient P.

The communication device 13 can comprise at least one communication interface 14 for establishment of the communication connection, wherein each communication interface 14 can be a wired and/or wireless communication interface. The communication can be carried out based on a standard communication protocol. For example, the communication interface can be a WLAN interface, a 5G interface, a USB interface, a Bluetooth interface, a WiFi interface or any other standard interface or an arbitrary combination thereof.

The device 11 comprises a supply apparatus 20 to which an instrument 21 operated by means of the supply apparatus 20 is connected or can be connected. Optionally, also multiple instruments 21 can be connected to the supply apparatus 20.

In the embodiment illustrated here by way of example electrosurgical instruments 21 are described. The device 11 can therefore be denoted as electrosurgical device. Additionally or alternatively, also other instruments 21 can be used, for example ultrasound instruments or cryo-instruments. Depending on the instrument type, the at least one operation medium is provided by supply apparatus 20, that means, for example, an electrical power and/or a fluid and/or ultrasound, etc.

The instrument 21 has a line 22 for connection with supply apparatus 20. Via this line 22 an electrical and/or optical and/or fluidical and/or ultrasound guiding connection can be established with supply apparatus 20. For this purpose, line 22 comprises a connector 23 at its end opposite the instrument 21, by means of which a releasable connection with a socket or another appropriate connection device of supply apparatus 20 can be established.

The electrosurgical instrument 21 can be configured as monopolar instrument, bipolar instrument or multipolar instrument, for example. Then it comprises at least one instrument electrode 24 that can be operated using an electrical voltage and/or an electrical current.

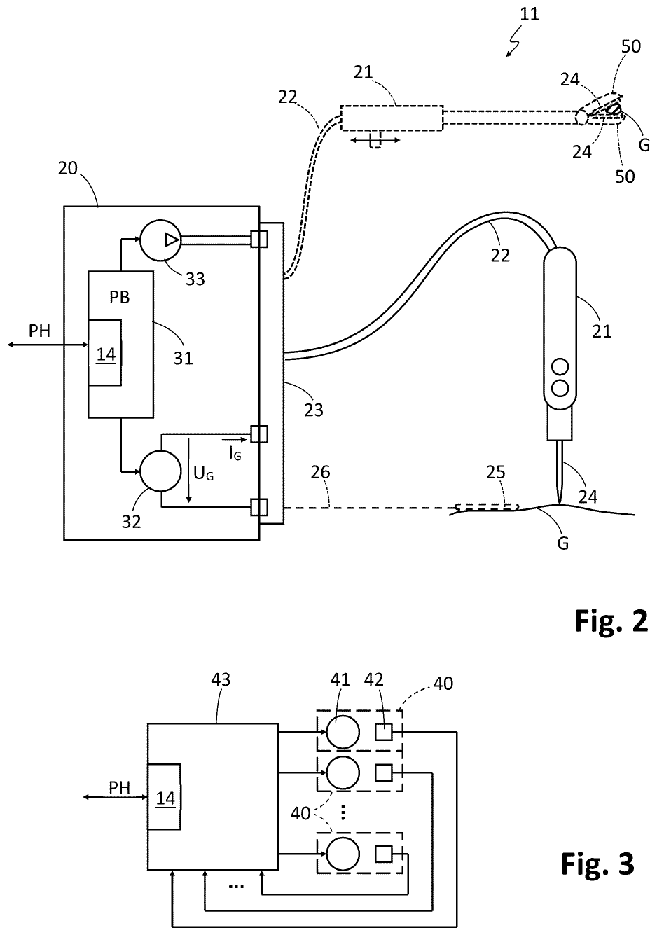

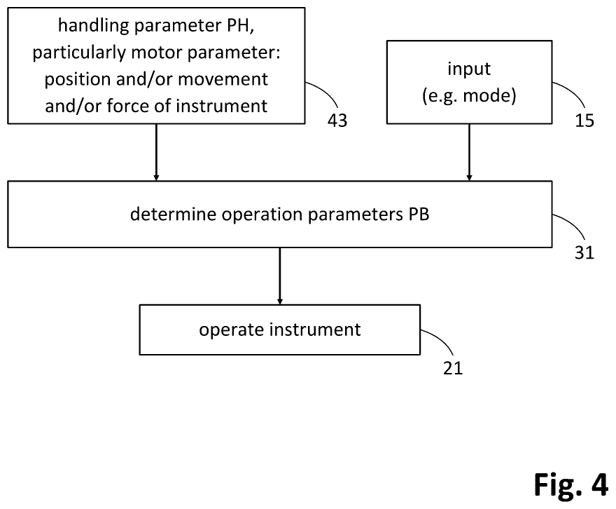

In a monopolar instrument or a bipolar or multipolar instrument operated as monopolar instrument the at least one electrosurgical device comprises additionally a neutral electrode 25, which can be omitted in instruments operating in bipolar or multipolar manner. The neutral electrode 25 is attached to the patient P in an electrically conductive manner for treatment of biological tissue G of the patient P (FIG. 1). Thereby a treatment circuit from supply apparatus 20 via the instrument electrode 24, the tissue G to be treated of the patient P and the neutral electrode 25 to the supply apparatus 20 can be closed (FIGS. 1 and 2). For this purpose, neutral electrode 25 is electrically connected via an electrode line 26 with supply apparatus 20.

In case of a bipolar or multipolar instrument 21 (illustrated in dashed lines in FIG. 2) the treatment circuit from supply apparatus 20 via one of the provided instrument electrodes, the tissue G to be treated of the patient P, another of the provided instrument electrodes to the supply apparatus 20 can be closed (without neutral electrode 25). The illustration in FIG. 2 is schematic. It is understood that each instrument 21 can be connected to supply device 20 via an individual connector 23.

The supply apparatus 20 has an electrical generator 32, which can be controlled by an apparatus control 31 and which supplies the instrument 21 with electrical energy. By means of generator 32 the instrument can be supplied with electrical power, which can be applied to the at least one instrument electrode 24, for example. The generator 32 can operate as impressed voltage source or as impressed current source. Using the generator 32 a high frequency generator voltage UG and/or a high frequency generator current IG can be provided to the connected instrument 21.

The supply apparatus 20 can optionally comprise a fluid source 33 that can be controlled by means of the apparatus control 31. In doing so, it is made possible to supply a connected electrosurgical instrument 21 for the operation with a fluid and particularly a gas, for example argon. For example, the gas can be used in the electrosurgical instrument 21 for producing a plasma for the tissue treatment. As mentioned, also other instrument types can be used.

The robot 12 has a movable or positionable robot arm 37. As apparent from the schematic illustration in FIG. 1, robot arm 37 comprises multiple arm parts 38 that can be moved relative to each other. Two directly adjacent arm parts 38 are pivotably supported on one another according to the example. The robot arm 37 or one of the arm parts 38 can be rotatably and/or pivotably arranged on a base 39 of robot 12, wherein the rotation axis can extend in vertical direction, according to the example.

For rotation and/or carrying out a pivot movement or another movement between the robot arm 37 and the base 39 or between two arm parts 38 movably connected with each other, robot 12 comprises multiple drive units 40. For this purpose, the drive units 40 can comprise a controllable electric motor 41 in each case. The drive units 40 can optionally comprise a motor sensor 42 assigned to the electric motor 41, for example a rotation position and/or rotational speed sensor.

The robot 12 comprises a robot control 43. The robot control 43 is configured to control the drive units 40 of robot arm 37. In doing so, the arm parts 38 can be moved and positioned relative to each other and/or the robot arm 37 can be moved and positioned relative to base 39. Signals of the motor sensors 42 can be optionally transmitted to robot control 43 for controlling the respective drive unit 40 if the respective drive unit 40 comprises a motor sensor 42. Additionally or alternatively, motor parameters can be preset and/or determined in the robot control 43, for example a motor voltage and/or a motor current and/or a motor torque and/or a motor rotational speed and/or a temporal change of any of the motor parameters of the respective drive unit 40 mentioned above.

On the end opposite base 39, robot arm 37 comprises a holding device 44 that is configured for holding or arranging an instrument or optionally also multiple instruments 21. The holding device 44 can be configured as gripping device, for example.

The holding device 44 and thus an instrument 21 that is held there can be moved and positioned relative to the patient P using the robot arm 37, so that the instrument 21 can be orientated relative to the tissue G to be treated. The movement of robot arm 37 and therefore of the instrument 21 held there can be controlled or influenced by an operator OP by means of the operation device 15 in the embodiment.

An instrument 21 is handled using the robot 12, whereby handling parameters PH control this handling in open-loop or closed-loop manner or describe this handling. The handling comprises the relative position and/or the relative speed between an instrument 21 and the tissue G to be treated and/or a force between the instrument 21 and the tissue G to be treated, which is exerted using the instrument 21 onto the tissue G to be treated. The at least one handling parameter PH is preset or determined by means of robot control 43 and can be, for example, at least one set point value and/or at least one actual value of the movement of the robot arm or can be derived therefrom. The at least one handling parameter PH particularly describes the control of the drive units 40 and can be an electrical motor parameter of the electric motor 41.

While robot control 43 controls the robot arm 37 according to the at least one handling parameter PH in open-loop or closed-loop manner, at least one operation parameter PB of the respective instrument 21 can be controlled in open-loop or closed-loop manner by means of apparatus control 31. The operation parameter PB is a parameter describing the provided operation media, for example an electrical and/or fluidical parameter that describes the operation of the respective electrosurgical instrument 21.

For example, as operation parameter PB, at least one of the following parameters can be controlled in open-loop or closed-loop manner by means of the apparatus control 31 in the supply apparatus 20:
a current parameter of the generator current IG, wherein the current parameter can be an amplitude, a frequency, a crest factor, a duty factor or duty cycle, a waveform or an arbitrary combination of these current parameters;
a voltage parameter of the generator voltage UG, wherein the voltage parameter can be an amplitude, a frequency, a crest factor, a duty factor or duty cycle, a waveform or an arbitrary combination of these voltage parameters;
a fluid parameter of a fluid supplied to the instrument, for example a pressure, a flow velocity, a volume flow rate, a mass flow rate or an arbitrary combination of these fluid parameters;
an activation duration during which a generator voltage UG and/or a generator current IG and/or an electrical power is provided for a connected instrument 21 continuously or in clocked manner;
an electrical power for the instrument, particularly for an electrode of the instrument.

The operation parameters PB distinguish from the handling parameters PH. While the operation parameters PB define a (for example electrical and/or fluidical) operation condition of a connected instrument 21, the handling parameters PH define the position (spatial position and/or orientation in the space) of an instrument 21 arranged on the holding device 44 of robot 12 relative to the tissue G to be treated of patient P. Additionally, the handling parameters PH can also describe a force (for example pressing force, clamping force, cutting force, forward movement force) between an instrument 21 and the tissue G to be treated.

By means of the communication device 13 or at least one communication interface 14 robot control 43 and apparatus control 31 are communicatively connected, wherein at least a transmission of communication data from robot control 43 to apparatus control 31 is made possible. The communication connection can also be bidirectional. Additionally or alternatively, also a communication connection between the operation device 15 on one hand and the apparatus control 31 and/or the robot control 43 on the other hand can be established using the communication device 13 as schematically illustrated in FIG. 1.

By means of the communication connection or the communication device 13 the at least one handling parameter PH or at least one of the handling parameters PH is provided to apparatus control 31. The apparatus control 31 is configured to determine the operation parameter PB or at least one of the operation parameters PB based on the handling parameter PH or at least one of the handling parameters PH. At least one of the operation parameters PB, multiple or all operation parameters PB are thus determined and controlled in open-loop or closed-loop manner depending on one or more handling parameters PH.

For example, an operation parameter PB or multiple operation parameters PB can be determined and controlled in open-loop or closed-loop manner depending on the relative speed of an instrument 21 relative to the tissue G and/or a force between the instrument 21 and the tissue G. Thereby particularly the electrical voltage and/or the electrical current and/or the electrical power at least at one of the instrument electrodes 24 is determined and accordingly controlled in open-loop or closed-loop manner depending on the speed or the force.

Specifically, the electrical operation of an electrosurgical instrument 21 can be changed or adapted depending on its relative speed or its relative force relative to the tissue G to be treated according to one or multiple of the possibilities described in the following. The change or adaption of the operation parameter PB or of at least one of the operation parameters PB can thereby be carried out in a stepless manner and/or continuously depending on the position and/or speed and/or force in each case. In the following explanation the relative speed relates to the speed of the electrosurgical instrument 21 relative to the tissue G to be treated and the force relates to a force applied between the electrosurgical instrument 21 and the tissue G to be treated (for example pressing force against tissue G, forward movement force during movement of the instrument along the surface of the tissue G or into the tissue G, clamping force between two movable instrument parts or cutting force during cutting of the tissue G):
1) An electrical power provided to the electrosurgical instrument 21 or the at least one instrument electrode 24 is increased with increasing relative speed, wherein the electrical voltage (particularly generator voltage UG) provided to the electrosurgical instrument 21 can be kept constant.
2) With increasing force, the electrical power can be increased that is provided to the at least one electrosurgical instrument 21 and particularly the instrument electrode 24. Thereby, for example, the voltage (particularly generator voltage UG) provided to the electrosurgical instrument 21 can be increased with increasing force. Preferably the crest factor of the generator voltage UG and/or the generator current IG can be reduced with increasing force.
3) An activation duration during which an electrical power is provided to the electrosurgical instrument 21 or the instrument electrode 24 continuously or in clocked manner can be reduced with increasing force, wherein the electrical power can be reduced particularly if the force describes a clamping force with which tissue G is clamped between two movable parts of the instrument (compare FIG. 2).
4) With increasing distance of two movable instrument parts of the instrument that act on and, for example, clamp tissue G to be treated (compare FIG. 2) an activation duration can be increased, while an electrical power is provided to the electrosurgical instrument 21 continuously or in clocked manner, whereby particularly the electrical power can be increased with increasing distance.

The two movable instrument parts of an instrument 21 can be, for example, jaws 50 of an electrosurgical instrument and particularly an electrosurgical instrument operating in a forceps-like manner (by way of example shown in FIGS. 1 and 2). The jaws 50 can be moved toward and away from each other and can be pivoted, for example. The relative distance is here described by an angle between the jaws and is large if the angle between the jaws is large, whereas the relative distance is small if the angle between the jaws is small.

The movable instrument parts or jaws 50 of (according to the example electrosurgical) instrument 21 arranged on the holding device 44 can be actuated by means of robot 12 or by means of holding device 44, for example in order to clamp tissue G between the jaws 50. On the jaws 50 one instrument electrode 24 can be provided in each case that is in contact with the tissue G to be treated if it is clamped between the jaws 50. In doing so, the tissue G can be coagulated, for example.

Due to the configuration according to the invention an operation parameter PB or multiple operation parameters PB can be quickly adapted to changing handling parameters PH. In doing so, the treatment of tissue G can be optimized. Operation parameters, for example electrical and/or fluidical operation parameters, can be very quickly adapted to changing movement speeds, forces or other handling parameters PH with which robot 12 acts on instrument 21. In doing so, an optimum influence of instrument 21 adapted to the movement and force parameters is guaranteed at any point in time.

Additionally or alternatively to the described embodiments, it is possible to determine a penetration depth and/or cutting depth of a needle, a knife or a needle-shaped instrument electrode 24 or a cutting electrode into the tissue G to be treated and to vary an operation parameter PB or multiple operation parameters PB depending on the penetration depth or cutting depth. For example, an electrical power and/or electrical voltage provided to the electrosurgical instrument 21 can be increased if the penetration depth or cutting depth increases. Additionally or alternatively, the crest factor can be reduced with increasing penetration depth and/or cutting depth.

The penetration depth or cutting depth can be determined, for example, in that first a contact between the distal end of instrument 21 (for example needle electrode or cutting electrode) with the tissue G is determined. For example, this can be carried out in that an operation parameter PB changes upon contact of the needle electrode or cutting electrode with tissue G, for example because a current flow through the tissue G to be treated is created thereby and/or a voltage applied to the instrument electrode 24 (needle electrode or cutting electrode) changes and particularly decreases. Starting from this reference position a movement carried out by means of robot 12 can be detected and the penetration depth or cutting depth into the tissue G to be treated can be described.

As described above, the different handling parameters PH can be determined without additional sensors on the instrument 21 using the parameters, particularly motor parameters, that are already known in the robot 12. Optionally in the robot 12 and particularly in the robot arm 37 sensors can be provided, such as the explained motor sensors 42. Such robot sensors are, however, not directly assigned to an instrument 21 arranged on the holding device 44 and are anyway provided in the robot 12 for the control of the robot arm 37. Additional sensors assigned to the instrument 21 and attached there or on the holding device 44 are not required and are not present in the embodiments.

The invention refers to a surgery system 10 as well as a method for its operation. The surgery system 10 has a device 11 and a robot 12. The device 11 comprises a supply apparatus 20 and at least one instrument 21 that is configured for treatment of biological tissue G and that is connected to the supply apparatus 20. The instrument 21 or at least one of the provided instruments 21 is arranged on a holding device 44 of robot 12 and can be positioned and/or moved relative to the tissue G to be treated, wherein this relative movement and/or relative position is described by means of at least one handling parameter PH. Additionally or alternatively, a handling parameter PH can describe a force between the instrument 21 and the tissue G. At least one of the handling parameters PH or multiple handling parameters PH are provided to device 11 via a communication device, that determines and controls in open-loop or closed-loop manner one or more operation parameters PB for the operation of the instrument 21 depending on one or more handling parameters PH. In doing so, an optimum adaption of the operation of the instrument 21 to the current movement or force exerted on the instrument 21 by robot 12 is achieved.

### LIST OF REFERENCE SIGNS

10 surgery system
11 device
12 robot
13 communication device
14 communication interface
15 operation device
20 supply apparatus
21 instrument
22 line
23 connector
24 instrument electrode
25 neutral electrode
26 electrode line
31 apparatus control
32 generator
33 fluid source
37 robot arm
38 arm part
39 base
40 drive unit
41 electric motor
42 motor sensor
43 robot control
44 holding device
50 jaw
G tissue
IG generator current
OP operator
P patient
PB operation parameter
PH handling parameter
UG generator voltage

1. A surgery system (10) comprising:
a device (11) having a supply apparatus (20) comprising an apparatus control (31) and having at least one instrument (21) that serves to treat biological tissue (G) and that is connected to the supply apparatus (20), wherein the apparatus control (31) is configured to determine and set at least one operation parameter (PB) for the operation of the at least one connected instrument (21);
a robot (12) having a robot control (43) and at least one movable robot arm (37)on which a holding device (44) for arranging the at least one instrument (21) is provided, wherein the robot control (43) is configured to control the robot (12) in order to handle the at least one instrument (21) arranged on the holding device (44), wherein at least one handling parameter (PH) predefined and/or determined by the robot (12) describes a movement and/or a position of the respective at least one instrument (21) relative to the biological tissue (G) and/or a force between the at least one instrument (21) and the biological tissue (G); and
a communication device (13) that is configured to provide the at least one handling parameter (PH) to the apparatus control (31), wherein the apparatus control (31) is configured to determine the at least one operation parameter (PB) based on the at least one handling parameter (PH).

2. The surgery system according to claim 1, wherein the communication device (13) comprises at least one communication interface (14).

3. The surgery system according to claim 2, wherein the at least one communication interface (14) is part of the apparatus control (31) and/or the robot control (43).

4. The surgery system according to claim 1, wherein the robot (12) comprises at least one drive unit (40) for positioning and/or moving the holding device (44), wherein the at least one drive unit (44) comprises an electric motor (41).

5. The surgery system according to claim 4, wherein at least one motor parameter of the electric motor (41) defines the at least one handling parameter (PH) or is used for determination of the at least one handling parameter (PH) in the robot control (43).

6. The surgery system according to claim 5, wherein the at least one motor parameter comprises at least one of the following parameters:
a motor voltage;
a motor current;
a motor torque;
a motor rotational speed;
a motor rotation angle; and
a temporal change of any above-indicated motor parameter.

7. The surgery system according to claim 1, wherein the robot (12) comprises at least one sensor (42), wherein the at least one sensor (42) is configured to provide a sensor value defining the at least one handling parameter (PH) or based on which the at least one handling parameter (PH) is determined in the robot control (43).

8. The surgery system according to claim 1, wherein the at least one operation parameter (PB) for the at least one instrument (21) is controlled depending on the at least one handling parameter (PH) which indicates a movement speed of the at least one instrument (21).

9. The surgery system according to claim 1, wherein the at least one operation parameter (PB) for the at least one instrument (21) is controlled depending on the at least one handling parameter (PH) which indicates the force between the at least one instrument (21) and the biological tissue (G) to be treated.

10. The surgery system according to claim 1, wherein the apparatus control (31) and/or the robot control (43) is configured to detect a contact between the at least one instrument (21) with the biological tissue (G) to be treated.

11. The surgery system according to claim 10, wherein the apparatus control (31) and/or the robot control (43) is configured to determine a position change of the at least one instrument (21) since the contact was made.

12. The surgery system according to claim 11, wherein the apparatus control (31) and/or the robot control (43) is configured to determine a penetration depth into the biological tissue (G) to be treated based on the position change of the at least one instrument (21).

13. The surgery system according to claim 1, wherein the apparatus control (31) and/or the robot control (43) is configured to determine a position of a movable part (50) of the at least one instrument (21).

14. A method for operating a surgery system (10), wherein the surgery system (10) comprises a device (11) having a supply apparatus (20) with an apparatus control (31) and having at least one instrument (21) for treating biological tissue (G) connected to the supply apparatus (20), wherein the surgery system (10) additionally comprises a robot (12) having a robot control (43) and having a movable robot arm (37) on which a holding device (44) for arranging the at least one instrument (21) is provided, and wherein the surgery system (10) additionally comprises a communication device (13), wherein the method comprises:
determining and setting at least one operation parameter (PB) for operation of the at least one connected instrument (21) by the apparatus control (31);
controlling the robot (12) by the robot control (43) for handling the at least one instrument (21) arranged on the holding device (44), wherein at least one handling parameter (PH) that is preset or determined by the robot (12) describes a movement and/or a position of the at least one instrument (21) relative to the biological tissue (G) and/or a force between the at least one instrument (21) and the biological tissue (G);
providing the at least one handling parameter (PH) to the apparatus control (31) by the communication device (13); and
determining the at least one operation parameter (PB) based on the at least one handling parameter (PH) by the apparatus control (31).
